(19) United States
(12) Patent Application Publication
(10) Pub. No.: US 2025/0034257 A1
(43) Pub. Date: Jan. 30, 2025

(54) STABLE HIGH CONCENTRATION ARGININE FORMULATIONS CONTAINING PD-1 ANTIBODY AND METHODS OF USE THEREOF

(71) Applicant: BeiGene Switzerland GmbH, Basel (CH)

(72) Inventors: Jun Wu, Suzhou (CN); Xiaoqing Jin, Suzhou (CN); Yu Ji, Suzhou (CN); Sufang Gu, Suzhou (CN); Jian Shen, Suzhou (CN); Bo Qiu, Guangzhou (CN)

(73) Assignee: BeiGene Switzerland GmbH, Basel (CH)

(21) Appl. No.: 18/913,543

(22) Filed: Oct. 11, 2024

Related U.S. Application Data
(63) Continuation of application No. PCT/CN2023/087866, filed on Apr. 12, 2023.

(30) Foreign Application Priority Data
Apr. 14, 2022 (WO) PCT/CN2022/086872

(51) Int. Cl. C07K 16/28

(52) U.S. Cl. CPC C07K 16/2818; C07K 2317/24

(57) ABSTRACT

The present invention relates generally to the field of pharmaceutical formulations of antibodies against human programmed death receptor PD-1, or antigen binding fragments thereof. The formulations may further contain a formulation buffer, a viscosity reducer, and a non-ionic surfactant. The pharmaceutical formulations of the present invention exhibit low viscosity and a substantial degree of antibody stability after being subjected to thermal and other physical stress. Also provided are methods of making and methods of using such antibody formulations.

### CROSS-REFERENCE TO RELATED APPLICATION(S)

This application is a continuation of International Patent Application No. PCT/CN2023/087866, filed Apr. 12, 2023, which claims priority from International Patent Application No. PCT/CN2022/086872, filed Apr. 14, 2022. The contents of these applications are incorporated herein by reference in their entireties.

### SEQUENCE LISTING

The instant application contains a Sequence Listing which has been submitted electronically in xml format, and is hereby incorporated by reference in its entirety. Said xml file was created on Oct. 10, 2024, is named 138881_0982_Sequence_Listing.xml, and is 17 bytes in size.

### FIELD OF THE DISCLOSURE

Disclosed herein are stable high concentration formulations comprising antibodies or antigen binding fragments thereof that binds to human programmed death receptor 1 (PD-1). Also disclosed herein are methods of preparing the formulations and treating cancers with the formulations of the present disclosure.

### BACKGROUND OF THE DISCLOSURE

Antibodies as therapeutics have seen increased use in the clinic. However, while antibodies generally have a similar structure, they are different in primary amino acid sequence, even for antibodies that bind to the same target protein. The characteristics of the primary amino acid sequence of the antibody is one of the major determinants of the properties of antibody solubility and/or stability in different formulations. An antibody formulation that provides for solubility and stability of one antibody can perform poorly for another antibody, resulting in antibody precipitation or fragmentation. This is especially true when a subcutaneous antibody formulation is desired.

Subcutaneous injection has gained increasing attention for the delivery of protein therapeutics due to its potential to provide for patient self-administration. With a fast, low-volume injection, the patient can administer the antibody therapeutic without the need for an intravenous infusion, which typically requires a hospital visit. However, many antibodies require a certain dose to be effective, generally requiring concentration of the antibody into a small volume. The volume limitation of subcutaneous route of administration is a critical factor to be considered for subcutaneous administration. leading to a need for highly concentrated antibody dose. In turn, this creates challenges relating to solubility, physical and chemical stability of the protein, difficulties of manufacture, storage, and delivery of the subcutaneous antibody formulation. For example, antibodies can lose solubility and form particulates in certain formulations during processing and/or storage, which renders the subcutaneous administration less effective. Due to the concentrated nature of the antibody in a subcutaneous formulation, high viscosity is another problem to overcome as it limits the injectability of the product. Also, in the manufacturing process, a highly viscous antibody formulation presents difficulties in processing, particularly in ultrafiltration and sterile filtration. Lastly, the subcutaneous antibody formulation needs to maintain the structure and function of the antibody. A subcutaneous antibody formulation that leads to proteolysis or degradation of the antibody structure will have reduced efficacy as well as one that impairs the antibody's ability to bind to its target protein.

Thus, there is a long felt need in the art for subcutaneous antibody formulations of anti-human PD-1 antibodies for treating various cancers and infectious diseases. Such formulations can have good antibody solubility, stability, a long shelf-life, and be amenable to administration at high concentrations.

### SUMMARY OF THE INVENTION

The invention provides a stable, low viscosity and high concentration antibody formulation. A low viscosity pharmaceutical formulation comprising:
about 10 mg/mL to about 200 mg/mL of an anti programmed death receptor 1 PD-1 antibody, or antigen binding fragment thereof;
a formulation buffer providing a pH of about 5.0 to about 7.0;
a viscosity reducer; and
a non-ionic surfactant, wherein mentioned formulation has a viscosity of no more than 35 cP, and an osmolarity from about 200 mOsmol/kg to about 400 mOsmol/kg.

The formulation wherein the PD-1 antibody or antigen binding fragment thereof, comprises (a) a HCDR1 (Heavy Chain Complementarity Determining Region 1) of SEQ ID NO: 1, (b) a HCDR2 of SEQ ID NO:2, (c) a HCDR3 of SEQ ID NO:3 and a light chain variable region that comprises: (d) a LCDR1 (Light Chain Complementarity Determining Region 1) of SEQ ID NO:4, (e) a LCDR2 of SEQ ID NO:5, and (f) a LCDR3 of SEQ ID NO:6.

The formulation wherein the formulation buffer is selected from the group consisting of histidine, acetate, citrate, succinate, phosphate, mixture of histidine and acetic acid, or mixture of histidine and citric acid

The formulation wherein the formulation buffer is histidine.

The formulation wherein the formulation buffer is acetate.

The formulation wherein the concentration of buffer is 15 mM to 25 mM.

The formulation wherein the formulation comprises 20 mM histidine buffer or 20 mM acetate buffer.

The formulation wherein the pH is 5.0-6.0.

The anti-human PD-1 antibody formulation wherein the viscosity reducer is an arginine salt.

The formulation wherein the arginine salt is an equal mixture of L-arginine and L-glutamic acid (ArgGlu) from 50 mM to 280 mM.

The formulation wherein the arginine salt is an L-arginine and L-glutamic acid complexed salt (ArgGlu) from 50 mM to 280 mM.

The formulation wherein the arginine salt is an equal mixture of L-arginine and L-aspartic acid (ArgAsp) from 50 mM to 280 mM.

The formulation wherein the arginine salt is an L-arginine and L-aspartic acid complexed salt (ArgAsp) from 50 mM to 280 mM.

The formulation wherein the arginine salt is L-arginine hydrochloride (ArgHCl) from 50 mM to 280 mM.

The formulation wherein the non-ionic surfactant is selected from the group consisting of polysorbate 20, polysorbate 80 or poloxamer188.

The formulation wherein the concentration of polysorbate 80 is from 0.02% to 0.08%.

The formulation wherein polysorbate 20 concentration is 0.05%.

The formulation wherein the formulation comprises 20 mM Histidine-Histidine HCl, 140 mM ArgGlu, 0.05% polysorbate 80 with a pH of pH5.5.

The formulation wherein the formulation comprises 20 mM Acetate, 140 mM ArgAsp, 0.05% polysorbate 80 with a pH of pH5.5.

The formulation wherein the formulation comprises 20 mM Histidine-Histidine HCl, 140 mM ArgHCl, 0.05% polysorbate 80 with a pH of pH5.5.

The formulation wherein the concentration of the anti-human PD-1 antibody, or antigen binding fragment thereof is from about 100 mg/mL to 200 mg/mL.

A method for treating cancer in a human patient in need thereof comprising subcutaneous administration of an effective amount of an anti-human PD-1 antibody formulation.

The method of claim 22, wherein the anti-human PD-1 antibody formulation is administered at a dose of about 100 mg to about 1000 mg.

The method wherein the anti-human PD-1 antibody formulation is subcutaneously administered at a dose of 200 mg.

The method wherein the anti-human PD-1 antibody formulation is subcutaneously administered at a dose of 300 mg.

The method wherein the anti-human PD-1 antibody formulation is subcutaneously administered at a dose of 400 mg.

The method wherein the anti-human PD-1 antibody formulation is subcutaneously administered at a dose of 500 mg.

The method wherein the anti-human PD-1 antibody formulation is subcutaneously administered once every three weeks.

The method wherein the anti-human PD-1 antibody formulation is administered once every week.

The method wherein the anti-human PD-1 antibody formulation is administered once every two weeks.

The method wherein the anti-human PD-1 antibody formulation is administered once every three weeks.

The method wherein the cancer is lung cancer (including small-cell lung cancer, or non-small cell lung cancer), adrenal cancer, liver cancer, stomach cancer, cervical cancer, melanoma, renal cancer, breast cancer, colorectal cancer, leukemia, bladder cancer, bone cancer, brain cancer, an endometrial cancer, head and neck cancer, lymphoma, ovarian cancer, skin cancer, thyroid tumor, or esophageal cancer.

The method wherein the human patient is administered at least one other therapeutic agent.

The method wherein the at least one other therapeutic agent is zanubrutinib, pamiparib, an anti-CTLA4 antibody, an anti-4-1BB antibody, an anti-OX40 antibody, an anti-TIGIT antibody, an anti-TIM-3 antibody, a second PD-1 antibody, a CD40 agonist, a TLR agonist, a CAR-T cell, or a chemotherapeutic agent.

In some embodiments, the antibody formulation comprises an anti-PD-1 antibody, or antigen binding fragment thereof, a formulation buffer, a viscosity reducer, and a non-ionic surfactant. In some embodiments, the formulation buffer provides a pH range of between 5.0 and 7.0. In some embodiments, the antibody formulation has a viscosity of no more than 50 centiPoise (cP). In some embodiments, the antibody formulation has an osmolarity of about 200 mOsmol/kg to about 400 mOsmol/kg. In some embodiments, the antibody formulation is stable upon freeze-thaw stress and 3-months storage at 5° C. and 25° C.

In some embodiments, the antibody formulation can comprise or consist essentially of between about 75 mg/mL to about 200 mg/mL anti-PD-1 antibody or antigen binding fragment thereof, a formulation buffer, a viscosity reducer, and a non-ionic surfactant, and has a pH of about 5.5±0.5. In some embodiments, the antibody formulation can consist essentially of about 100 mg/mL to about 200 mg/mL anti-PD-1 antibody, a formulation buffer, a viscosity reducer, and a non-ionic surfactant, and has a pH of 5.5±0.5.

In some embodiments, the formulation buffer is selected from the group consisting of histidine, acetate, citrate, succinate, phosphate, mixture of histidine and acetic acid, mixture of histidine and citric acid. In some embodiments, the formulation buffer can be histidine buffer. In some embodiments, the concentration of histidine buffer is from about 10 mM to about 30 mM. In some embodiments, the concentration of the histidine buffer is about 20 mM histidine.

In other embodiments, the formulation buffer is acetate buffer. In some embodiments, the concentration of acetate buffer is about 5 mM to about 30 mM, preferably about 20 mM.

In some embodiments, the viscosity reducer is an amino acid or its derivatives. In some embodiments, the amino acid or its derivatives is L-arginine, arginine hydrochloride (ArgHCl), arginine acetate, arginine citrate, arginine succinate, arginine phosphate, arginine sulfate, arginine glutamate, arginine aspartate, L-proline, lysine hydrochloride, sodium glutamate (NaGlu), histidine glutamate or histidine hydrochloride. In some embodiments, the amino acid or its derivatives is arginine hydrochloride, arginine acetate, arginine citrate, arginine glutamate, arginine aspartate, lysine hydrochloride, preferably arginine hydrochloride, arginine glutamate and arginine aspartate.

In some embodiments, the concentration of amino acid or its derivatives is about 25 mM to about 400 mM. In some embodiments, the concentration of amino acid or its derivatives is about 50 mM to about 280 mM, preferably about 100 mM, about 105 mM, about 110 mM, about 115 mM, about 120 mM, about 125 mM, about 130 mM, about 135 mM and about 140 mM.

In some embodiments, the concentration of polysorbate is from about 0.01 to about 1.0 mg/mL, preferably about 0.2 mg/mL, about 0.3 mg/mL, about 0.4 mg/mL or about 0.5 mg/mL. In some embodiments, the polysorbate is polysorbate 80 or polysorbate 20, preferably polysorbate 80.

In some embodiments, the non-ionic surfactant is selected from the group consisting of polysorbate 80 (PS80), polysorbate 20 (PS20) or poloxamer188. In some embodiments, the concentration of non-ionic surfactant can be from about 0.01 to about 1 mg/mL. In some embodiments, the concentration of polysorbate is from about 0.01 to about 1.0 mg/mL, about 0.2 mg/mL, about 0.3 mg/mL, about 0.4 mg/mL, about 0.5 mg/mL, about 0.6 mg/mL, about 0.7 mg/mL, or about 0.8 mg/mL. In some embodiments, the polysorbate is polysorbate 80.

In some embodiments, the antibody formulation consists of about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 190 mg/mL or about 200 mg/mL, of an anti-PD-1 antibody, or antigen binding fragment thereof.

In some embodiments, the pharmaceutical compositions comprise a) about 150 mg/mL to about 200 mg/mL an anti-human PD-1 antibody, or antigen binding fragment thereof; b) about 20 mM histidine buffer or about 20 mM acetate buffer; c) about 100 mM to about 140 mM arginine hydrochloride; d) about 0.2 to about 0.5 mg/mL polysorbate 80; wherein the pH of mentioned pharmaceutical composition is about 5.5±0.5. In a specific embodiment, the arginine hydrochloride is a complex salt of arginine hydrochloride or a mixture of L-arginine and hydrochloric acid.

In another embodiments, the pharmaceutical compositions comprise a) about 150 mg/mL to about 200 mg/mL an anti-human PD-1 antibody, or antigen binding fragment thereof; b) about 20 mM histidine buffer or about 20 mM acetate buffer; c) about 100 mM to about 140 mM arginine glutamate; d) about 0.2 to about 0.5 mg/mL polysorbate 80; wherein the pH of mentioned pharmaceutical composition is about 5.5±0.5. In a specific embodiment, the arginine glutamate is a complex salt of arginine glutamate or a mixture of L-arginine and L-glutamic acid.

In another embodiments, the pharmaceutical compositions comprise a) about 150 mg/mL to about 200 mg/mL an anti-human PD-1 antibody, or antigen binding fragment thereof; b) about 20 mM histidine buffer or about 20 mM acetate buffer; c) about 100 mM to about 140 mM arginine aspartate; d) about 0.2 to about 0.5 mg/mL polysorbate 80; wherein the pH of mentioned pharmaceutical composition is about 5.5±0.5. In a specific embodiment, the arginine aspartate is a complex salt of arginine aspartate or a mixture of L-arginine and L-aspartic acid.

In some embodiments, the composition may have a viscosity of less than about 50 cP, less than about 40 cP, preferably less than about 35 cP at 25° C.

In some embodiments of the invention the PD-1 antibody is Tislelizumab (BGB-A317) or an antigen binding fragment of Tislelizumab.

Also provided herein are methods of treating cancer in a human patient who has cancer, comprising subcutaneous administration to the patient an effective amount of the antibody formulation as described herein.

Provided herein are methods of treating cancer in a human patient who has a PD-L1 expressing cancer, comprising subcutaneous administration to the patient an effective amount of the antibody formulation as described herein.

In some embodiments, the disclosure provides for methods of treating cancer with an anti-PD-1 subcutaneous antibody formulation in combination with another therapeutic agent. The other therapeutic agent is, for example, zanubrutinib, pamiparib, sitravatinib, an anti-CTLA4 antibody, an anti-4-1BB antibody, an anti-OX40 antibody, an anti-TIGIT antibody, an anti-TIM-3 antibody, a second PD-1 antibody, a CD40 agonist, a TLR agonist, a CAR-T cell, or a chemotherapeutic agent.

### BRIEF DESCRIPTION OF THE DRAWINGS

FIG. 1 shows the amounts of aggregates (FIG. 1A) and monomer (FIG. 1B) of each of the formulations as measured by SEC-HPLC. “T0” refers to the initial point of samples. “40C4W” refers to the samples stored at 40° C. for 4 weeks. “3 FT” refers to the samples subjected to three freeze/thaw cycles. “25C3M” refers to the samples stored at 25° C. for 3 months. “5C3M” refers to the samples stored at 5° C. for 3 months. “25C6M” refers to the samples stored at 25° C. for 6 months. “5C6M” refers to the samples stored at 5° C. for 6 months.

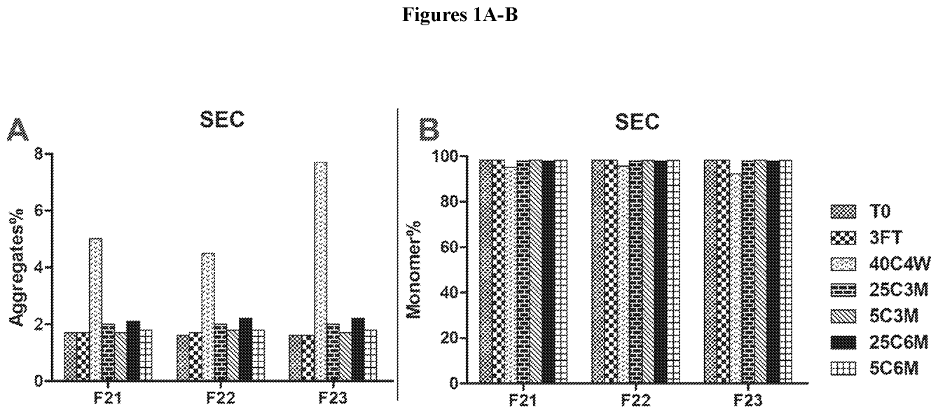

FIG. 2 shows the results of an CZE study of the formulations. “T0” refers to the initial point of samples. “40C4W” refers to the samples stored at 40° C. for 4 weeks. “25C3M” refers to the samples stored at 25° C. for 3 months. “5C3M” refers to the samples stored at 5° C. for 3 months. “25C6M” refers to the samples stored at 25° C. for 6 months. “5C6M” refers to the samples stored at 5° C. for 6 months.

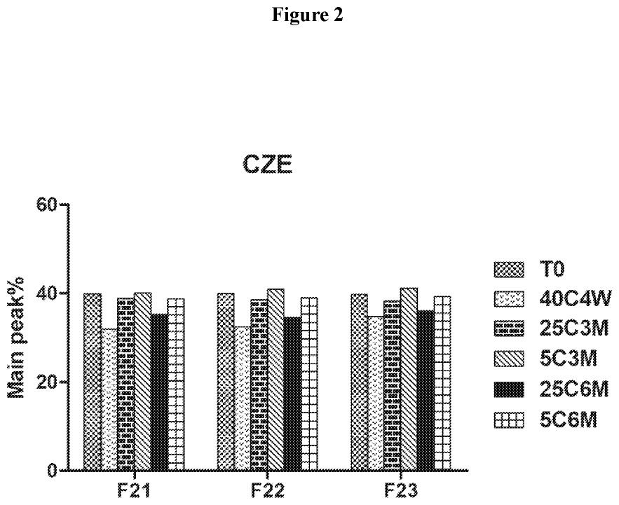

FIG. 3 shows the purity in the formulations as measured by CE-SDS under non-reduced conditions “T0” refers to the initial point of samples. “40C4W” refers to the samples stored at 40° C. for 4 weeks. “25C3M” refers to the samples stored at 25° C. for 3 months. “5C3M” refers to the samples stored at 5° C. for 3 months “25C6M” refers to the samples stored at 25° C. for 6 months. “5C6M” refers to the samples stored at 5° C. for 6 months.

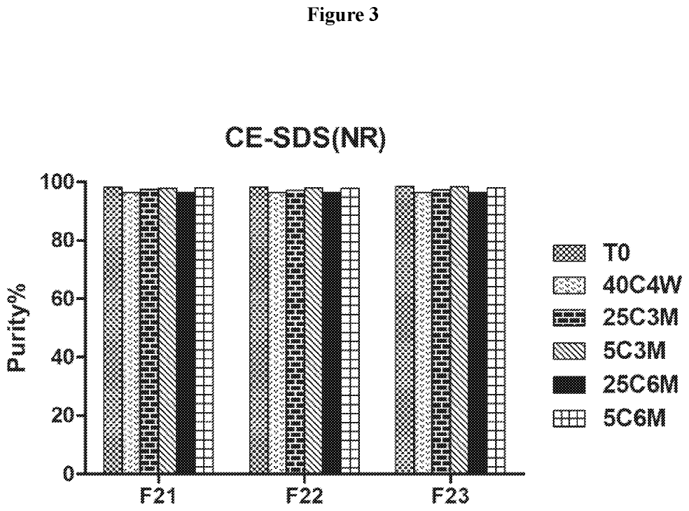

### DETAILED DESCRIPTION OF THE DISCLOSURE

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the” include their corresponding plural references unless the context clearly dictates otherwise.

The term “or” is used to mean, and is used interchangeably with, the term “and/or” unless the context clearly dictates otherwise.

Throughout this specification and the claims which follow, unless the context requires otherwise, the word “comprise,” and variations such as “comprises” and “comprising,” will be understood to imply the inclusion of a stated amino acid sequence, DNA sequence, step or group thereof, but not the exclusion of any other amino acid sequence, DNA sequence, step. When used herein the term “comprising” can be substituted with the term “containing,” “including” or sometimes “having.”

The terms “administration,” “administering,” “treating,” and “treatment” herein, when applied to an animal, human, experimental subject, cell, tissue, organ, or biological fluid, means contact of an exogenous pharmaceutical, therapeutic, diagnostic agent, or antibody formulation to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both.

The term “therapeutically effective amount” as herein used, refers to the amount of an anti-PD-1 antibody that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder, or a condition. In some embodiment of present disclosure, the subject is a human.

“Pharmaceutical formulation” or “formulation” refers to antibody preparations which are in such form as to allow the active ingredients to be effective, and which contain no additional components that would be toxic to the subjects to which the formulation would be administered.

A “stable” formulation is one in which the antibody is prepared in such a way as to preserve the antibody's physical stability and/or chemical stability and/or biological activity over time. Various analytical techniques for measuring protein stability are available in the art and are reviewed in Peptide and Protein Drug Delivery, 247-301, Vincent Lee Ed, Marcel Dekker, Inc, New York, N. Y., Pubs. (1991) and Jones, A. Adv. Drug Delivery Rev. 10:29-90 (1993). Stability can be measured at a selected temperature for a selected time period.

The term “antibody” herein is used in the broadest sense and specifically covers antibodies (including full length monoclonal antibodies) and antibody fragments so long as they recognize antigen, e.g., PD-1. An antibody is usually monospecific, but may also be described as idiospecific, heterospecific, or polyspecific. Antibody molecules bind by means of specific binding sites to specific antigenic determinants or epitopes on antigens.

The term “monoclonal antibody” or “mAb” or “Mab” herein means a population of substantially homogeneous antibodies, i.e., the antibody molecules comprised in the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) may be obtained by methods known to those skilled in the art. See, for example Kohler G et al., Nature 1975 256:495-497; U.S. Pat. No. 4,376,110; Ausubel F M et al., CURRENT PROTOCOLS IN MOLECULAR BIOLOGY 1992; Harlow E et al., ANTIBODIES: A LABORATORY MANUAL, Cold spring Harbor Laboratory 1988; and Colligan J E et al., CURRENT PROTOCOLS IN IMMUNOLOGY 1993. The mAbs disclosed herein may be of any immunoglobulin class including IgG, IgM, IgD, IgE, IgA, and any subclass thereof. A hybridoma producing a mAb may be cultivated in vitro or in vivo. High titers of mAbs can be obtained by in vivo production where cells from the individual hybridomas are injected intraperitoneally into mice, such as pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as a, 8, &, Y, or u, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains sequentially comprise FR-1 (or FR1), CDR-1 (or CDR1), FR-2 (FR2), CDR-2 (CDR2), FR-3 (or FR3), CDR-3 (CDR3), and FR-4 (or FR4). The assignment of amino acids to each domain is, generally, in accordance with the definitions of Sequences of Proteins of Immunological Interest, Kabat, et al., National Institutes of Health, Bethesda, Md. 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al, (1987) J Mol. Biol. 196:901-917 or Chothia, et al., (1989) Nature 342:878-883.

The term “hypervariable region” means the amino acid residues of an antibody that are responsible for antigen-binding. The hypervariable region comprises amino acid residues from a “CDR” (i.e., VL-CDR1, VL-CDR2 and VL-CDR3 in the light chain variable domain and VH-CDR1, VH-CDR2 and VH-CDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196:901-917 (defining the CDR regions of an antibody by structure). The term “framework” or “FR” residues mean those variable domain residues other than the hypervariable region residues defined herein as CDR residues.

Unless otherwise indicated, “antibody fragment” or “antigen-binding fragment” means antigen binding fragments of antibodies, i.e., antibody fragments that retain the ability to bind specifically to the antigen bound by the full-length antibody, e.g., fragments that retain one or more CDR regions. Examples of antigen binding fragments include, but not limited to, Fab, Fab′, F(ab′) 2, and Fv fragments; diabodies; linear antibodies; single-chain antibody molecules, e.g., single chain Fv (ScFv); nanobodies and multispecific antibodies formed from antibody fragments.

An antibody that binds to a specified target protein with specificity is also described as specifically binding to a specified target protein. This means the antibody exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies or binding fragments thereof, useful in the present invention will bind to the target protein with an affinity that is at least two-fold greater, preferably at least 10-times greater, more preferably at least 20-times greater, and most preferably at least 100-times greater than the affinity with non-target proteins. An antibody herein is said to bind specifically to a polypeptide comprising a given amino acid sequence.

The term “human antibody” herein means an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” means an antibody that comprises only mouse or rat immunoglobulin protein sequences, respectively.

The term “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The antibody of the present application has potential therapeutic uses in treating cancer. The term “cancer” or “tumor” herein means or describes the physiological condition in mammals that is typically characterized by unregulated cell growth. Examples of cancer include but are not limited to, lung cancer (including small-cell lung cancer, or non-small cell lung cancer), adrenal cancer, liver cancer, stomach cancer, cervical cancer, melanoma, renal cancer, breast cancer, colorectal cancer, leukemia, bladder cancer, bone cancer, brain cancer, an endometrial cancer, head and neck cancer, lymphoma, ovarian cancer, skin cancer, thyroid tumor, or esophageal cancer.

Further, the antibody of the present application has potential therapeutic uses in controlling viral infections and other human diseases that are mechanistically involved in immune tolerance or “exhaustion.” In the context of the present application, the term “exhaustion” refers to a process which leads to a depleted ability of immune cells to respond to a cancer or a chronic viral infection.

### Anti-PD-1 Antibody

The present disclosure provides for anti-PD-1 antibodies and subcutaneous formulations thereof. For example, Tislelizumab (BGB-A317), is an anti-PD-1 antibody disclosed in U.S. Pat. No. 8,735,553 with the sequences provided in Table 1 below.

TABLE 1
BGB-A317 (Tislelizumab)

| Domain | SEQ ID NO: | Amino Acid Sequence |
|---|---|---|
| HCDR1 | SEQ ID NO: 1 | GFSLTSYGVH |
| HCDR2 | SEQ ID NO: 2 | VIYADGSTNYNPSLKS |
| HCDR3 | SEQ ID NO: 3 | ARAYGNYWYIDV |
| LCDR1 | SEQ ID NO: 4 | KSSESVSNDVA |
| LCDR2 | SEQ ID NO: 5 | YAFHRFT |
| LCDR3 | SEQ ID NO: 6 | HQAYSSPYT |
| VH | SEQ ID NO: 7 | QVQLQESGPGLVKPSETLSLTCTVSGFSLTSYGVHWIRQPP |
| | | GKGLEWIGVIYADGSTNYNPSLKSRVTISKDTSKNQVSLKL |
| | | SSVTAADTAVYYCARAYGNYWYIDVWGQGTTVTVSS |
| VL | SEQ ID NO: 8 | DIVMTQSPDSLAVSLGERATINCKSSESVSNDVAWYQQKP |
| | | GQPPKLLINYAFHRFTGVPDRFSGSGYGTDFTLTISSLQAE |
| | | DVAVYYCHQAYSSPYTFGQGTKLEIK |
| IgG4 | SEQ ID NO: 9 | ASTKGPSVFPLAPCSRSTSESTAALGCLVKDYFPEPVTVSW |
| constant | | NSGALTSGVHTFPAVLQSSGLYSLSSVVTVPSSSLGTKTYT |
| domain | | CNVDHKPSNTKVDKRVESKYGPPCPPCPAPPVAGGPSVFL |
| | | FPPKPKDTLMISRTPEVTCVVVAVSQEDPEVQFNWYVDGV |
| | | EVHNAKTKPREEQFNSTYRVVSVLTVVHQDWLNGKEYKC |
| | | KVSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSKLTVDKSRWQEGNVFSCSVMHEALHNHYTQKSLS |
| | | LSLGK |
| Heavy | SEQ ID NO: 10 | QVQLQESGPGLVKPSETLSLTCTVSGFSLTSYGVHWIRQPP |
| Chain | | GKGLEWIGVIYADGSTNYNPSLKSRVTISKDTSKNQVSLKL |
| | | SSVTAADTAVYYCARAYGNYWYIDVWGQGTTVTVSSAST |
| | | KGPSVFPLAPCSRSTSESTAALGCLVKDYFPEPVTVSWNSG |
| | | ALTSGVHTFPAVLQSSGLYSLSSVVTVPSSSLGTKTYTCNV |
| | | DHKPSNTKVDKRVESKYGPPCPPCPAPPVAGGPSVFLFPPK |
| | | PKDTLMISRTPEVTCVVVAVSQEDPEVQFNWYVDGVEVH |
| | | NAKTKPREEQFNSTYRVVSVLTVVHQDWLNGKEYKCKVS |
| | | NKGLPSSIEKTISKAKGQPREPQVYTLPPSQEEMTKNQVSL |
| | | TCLVKGFYPSDIAVEWESNGQPENNYKTTPPVLDSDGSFFL |
| | | YSKLTVDKSRWQEGNVFSCSVMHEALHNHYTQKSLSLSL |
| | | GK |
| Light | SEQ ID NO: 11 | DIVMTQSPDSLAVSLGERATINCKSSESVSNDVAWYQQKP |
| chain | | GQPPKLLINYAFHRFTGVPDRFSGSGYGTDFTLTISSLQAE |
| | | DVAVYYCHQAYSSPYTFGQGTKLEIKRTVAAPSVFIFPPSD |
| | | EQLKSGTASVVCLLNNFYPREAKVQWKVDNALQSGNSQE |
| | | SVTEQDSKDSTYSLSSTLTLSKADYEKHKVYACEVTHQGL |
| | | SSPVTKSFNRGEC |

Anti-PD1 antibodies can include, without limitation, Tislelizumab, Pembrolizumab or Nivolumab. Tislelizumab is disclosed in U.S. Pat. No. 8,735,553. Pembrolizumab (formerly MK-3475), as disclosed by Merck, in U.S. Pat. Nos. 8,354,509 and 8,900,587 is a humanized lgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human lgG4-K monoclonal antibody. Nivolumab (clone 5C4) is disclosed in US Patent No. U.S. Pat. No. 8,008,449 and WO 2006/121168. Nivolumab is approved for the treatment of melanoma, lung cancer, kidney cancer, and Hodgkin's lymphoma.

### Further Alteration of the Framework of Fc Region

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered Clq binding and/or reduced or abolished complement dependent cytotoxicity (CDC). This approach is described in, e.g., U.S. Pat. No. 6,194,551 by Idusogie et al.

In yet another aspect, one or more amino acid residues are altered to thereby alter the ability of the antibody to fix complement. This approach is described in, e.g., the PCT Publication WO94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the PCT Publication WO00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001).

In still another aspect, the glycosylation of an antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such a glycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). PCT Publication WO 99/54342 by Umana et al., describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al., 2010 MAbs, 2:181-189). On the other hand, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19). Reduced ADCC can be achieved by operably linking the antibody to IgG4 engineered with combinations of alterations to have reduced or null FcγR binding or C1q binding activities, thereby reducing or eliminating ADCC and CDC effector functions. Considering physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and YFc region were reported to have impact on antibody interaction with Fcγ receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate PD-1 antibodies with low ADCC, CDC and instability, it is possible to modify the hinge and Fc region of human IgG4 and introduce a number of alterations. These modified IgG4 Fc molecules can be found disclosed in SEQ ID NOs: 83-88, U.S. Pat. No. 8,735,553.

### Methods of Treatment

The antibodies or antigen-binding fragments of the present disclosure are useful in a variety of applications including, but not limited to, methods for the treatment of an PD-1-associated disorder or disease. In one aspect, the PD-1-associated disorder or disease is cancer.

In one aspect, the present disclosure provides a method of treating cancer. In certain aspects, the method comprises administering to a patient in need an effective amount of an anti-PD-1 antibody or antigen-binding fragment. The cancer can include, without limitation, lung cancer (including small-cell lung cancer, or non-small cell lung cancer), adrenal cancer, liver cancer, stomach cancer, cervical cancer, melanoma, renal cancer, breast cancer, colorectal cancer, leukemia, bladder cancer, bone cancer, brain cancer, an endometrial cancer, head and neck cancer, lymphoma, ovarian cancer, skin cancer, thyroid tumor, or esophageal cancer.

An antibody or antigen-binding fragment of the invention can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antigen-binding fragment of the invention will depend on the type of disease to be treated, the type of antibody, the severity and course of the disease, whether the antibody is administered for preventive or therapeutic purposes, previous therapy, the patient's clinical history and response to the antibody, and the discretion of the attending physician.

Antibodies that are directed to PD-1 have been shown to be safe when administered to human cancer patients in various dose ranges and administration cycles. The subcutaneous antibody formulations disclosed herein can be administered at 100 mg, 200 mg, 300 mg, 400 mg, 500 mg, 600 mg, 700 mg, 800 mg, 900 mg or 1000 mg. The subcutaneous antibody formulation can be administered twice per day, daily, once per week, twice per week, three times per week, four times per week, five times per week, once every two weeks, once every three weeks, once every month, once every two months, once every three months, once every four months, once every five months or once every six months. In some embodiments, the dosing regimen comprises administering Tislelizumab at 100 mg once every three weeks. In some embodiments, the dosing regimen comprises administering Tislelizumab at 200 mg once every three weeks. In some embodiments, the dosing regimen comprises administering Tislelizumab at 300 mg once every three weeks. In some embodiments, the dosing regimen comprises administering Tislelizumab at 400 mg once every three weeks. In some embodiments, the dosing regimen comprises administering Tislelizumab at 500 mg once every three weeks. In some embodiments, the dosing regimen comprises administering Tislelizumab at 600 mg once every three weeks.

In certain embodiments, Tislelizumab can be administered in combination with other therapies, for example, zanubrutinib, pamiparib, an anti-CTLA4 antibody, an anti-4-1BB antibody, an anti-OX40 antibody, an anti-TIGIT antibody, an anti-TIM-3 antibody, a CD40 agonist, a TLR agonist, a CAR-T cell, or a chemotherapeutic agent.

### Pharmaceutical Compositions and Formulations

Also provided are compositions, including pharmaceutical formulations, comprising an anti-PD-1 antibody or antigen-binding fragment thereof, or polynucleotides comprising sequences encoding an anti-PD-1 antibody or antigen-binding fragment. In certain embodiments, compositions comprise one or more antibodies or antigen-binding fragments that bind to PD-1, or one or more polynucleotides comprising sequences encoding one or more antibodies or antigen-binding fragments that bind to PD-1. These compositions can further comprise suitable carriers, such as pharmaceutically acceptable excipients including buffers.

Pharmaceutical formulations of an anti-PD-1 antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), polysorbate 20 or polysorbate 80.

Sustained-release preparations can be prepared. Suitable examples of sustained-release preparations include semipermeable matrices of solid hydrophobic polymers containing the antibody, which matrices are in the form of shaped articles, e.g., films or microcapsules.

The formulations to be used for in vivo administration are generally sterile. Sterility can be readily accomplished, e.g., by filtration through sterile filtration membranes.

### EXAMPLES

The examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entirety.

### Analytical Methods

This methods section provides a summary of the methods used in the following Examples 1-5.

### Protein Concentration

Protein concentrations are determined spectroscopically using SoloVPE (C Technologies Inc.) based on variable path length and Beer-Lambert Law (A=cle, where A=absorbance, c=concentration, 1=path length, and e=1.5 mg ml⁻¹ cm₋₁). Samples are equilibrated to ambient temperature, added to a sample vessel (C Tech Inc. #OC0009-1-P50) and loaded into the vessel holder in the detection window platform. Per sample, a clean fibrette (#OF0002-P50) is installed into the fibrette coupler, and the slope is determined at 280 nm.

### Viscosity

The viscosity of antibody formulations is measured on a chip-based microVISC™ instrument (Rheosense), in which the pressure difference correlates with solution dynamic viscosity. Sample size is approximately 70-100 μL. Aliquots are loaded into a 400 μL microVISC™ disposable pipette and connected to the chip. Triplicate measurements are taken at a shear rate of 500 S⁻¹ and at a temperature of about 25° C.

### Turbidity

UV absorption at 350 nm is measured using 96 well plate Molecular Devices M2e™ reader as an indication of turbidity. The absorption readings are controlled against blank well reading and normalized for sample pathlength.

### SEC-HPLC

Formation of soluble aggregates is analyzed by size exclusion chromatography (SEC) on a Waters HPLC system. 100 g of protein is separated based on molecular size on a TSKgel G3000™ SWXL column maintained at 37±5° C. using an isocratic gradient. Molecular weight species are eluted and detected by UV absorption at 280 nm. The distribution of aggregates, monomer and fragments are quantitated via the peak areas for standards and samples.

### CZE

The charge heterogeneity of sample is determined using PA800 Plus™ (Beckman) by a capillary zone electrophoresis method (CZE) also known as free solution capillary electrophoresis. Samples are separated based on their electrophoretic mobilities caused by differences in charge and hydrodynamic radius of the analytes in a capillary filled with a buffer solution mainly consisting of caproic acid. The samples are analyzed in their native state when an external electric field is applied resulting in a specific peak pattern showing the various charge variants of the antibody (acidic, basic and main charge variants). Samples are injected by pressure and the mobilized proteins are detected by UV absorbance at 214 nm.

### CE-SDS (NR)

The purity of sample is determined using PA800 Plus™ (Beckman) by a capillary gel electrophoresis (CE) method. Samples are denatured with sodium dodecyl sulphate (SDS) and separated based on size in a capillary filled with a gel that acts as a sieving medium. In non-reduced (NR) samples, an alkylating agent, N-Ethylmaleimide (NEM), is added to avoid any fragmentation induced by sample preparation and to ensure that the main IgG peak remains intact. Samples are injected electrokinetically, and the mobilized proteins are detected by UV absorbance at 200 nm using a UV detector. The reportable value for non-reduced samples is the time corrected area percent (TCA %) of the IgG main peak.

### Example 1: Preparation of High Concentration Anti-PD-1 Antibody Formulations

An anti-human PD-1 antibody, Tislelizumab (BGB-A317, Table 1) was prepared and purified.

### Formula 1 (F1) Preparation:

Tislelizumab antibody was buffer exchanged into 20 mM Histidine-Histidine HCl pH6.0, and then concentrated to approximately 200 mg/mL with 30 kDa Amicon Ultra™ centrifugal filters. Varying concentrations of samples were made by dilution with 20 mM Histidine-Histidine HCl pH6.0 buffer.

### Formulas 2-7 (F2-F7) and Formulas 10-12 (F10-F12) Preparation:

To prepare Tislelizumab stock solutions, Tislelizumab was buffer exchanged into 20 mM Histidine-Histidine HCl pH6.0 with 10 kDa MWCO dialysis cassettes. Stock solutions of ArgGlu (a mixture of L-arginine and L-glutamic acid), ArgHCl, ArgAsp (a mixture of L-arginine and L-aspartic acid), Proline, LysHCI, NaGlu (an equimolar mixture of L-glutamic acid and sodium hydroxide), ArgGlu and trehalose combination, ArgHCl and trehalose combination and ArgHCl and Proline combination dissolved in 20 mM Histidine-Histidine HCl pH6.0, were prepared. All the stock solutions were adjusted to pH6.0 with hydrochloric acid. All the stock solutions were spiked into Tislelizumab stock solutions to achieve the desired excipient concentrations shown in Table 2. Samples were then concentrated to about 200 mg/mL with 30 kDa Amicon Ultra™ centrifugal filters. Formulations with varying protein concentrations were made by dilution with 1× stock solutions.

### Formulas 8 and 9 (F8 and F9) Preparation:

To prepare Tislelizumab stock solutions, Tislelizumab was buffer exchanged into 20 mM Histidine-Acetic acid pH6.0 and 20 mM Histidine-Citric acid pH6.0 with 10 kDa MWCO dialysis cassettes. Stock solutions of 500 mM (5×) Arginine dissolved in 20 mM Histidine-Acetic acid pH6.0 and 20 mM Histidine-Citric acid pH6.0 were prepared. pH was adjusted to 6.0 using Acetic acid and Citric acid. 5× Arginine stock solutions were spiked into Tislelizumab stock solutions of 20 mM Histidine-Acetic acid (pH6.0) and 20 mM Histidine-Citric acid (pH6.0). Subsequently, samples were concentrated to about 200 mg/mL with 30 kDa Amicon Ultra™ centrifugal filters. Formulations with varying protein concentrations were made by dilution with 100 mM (1×) Arginine stock solutions.

This preparation method can also be applied to the preparation of other high concentration anti-PD-1 antibody formulations.

TABLE 2
Formulation Compositions

| Formulation | pH | Buffer | Viscosity Reducer |
|---|---|---|---|
| F1 | 6.0 | 20 mM Histidine- | None |
| | | Histidine HCl | |
| F2 | 6.0 | 20 mM Histidine- | 140 mM Arginine + 140 mM |
| | | Histidine HCl | Glutamic acid (140 mM ArgGlu) |
| F3 | 6.0 | 20 mM Histidine- | 140 mM ArgHCl |
| | | Histidine HCl | |
| F4 | 6.0 | 20 mM Histidine- | 140 mM Arginine+ 140 mM |
| | | Histidine HCl | Aspartic Acid (140 mM ArgAsp) |
| F5 | 6.0 | 20 mM Histidine- | 250 mM Proline |
| | | Histidine HCl | |
| F6 | 6.0 | 20 mM Histidine- | 140 mM LysHCl |
| | | Histidine HCl | |
| F7 | 6.0 | 20 mM Histidine- | 140 mM NaGlu |
| | | Histidine HCl | |
| F8 | 6.0 | 20 mM Histidine- | 140 mM Arginine + Acetic acid |
| | | Acetic acid | (adjusting pH to 6.0) |
| F9 | 6.0 | 20 mM Histidine- | 140 mM Arginine + Citric acid |
| | | Citric acid | (adjusting pH to 6.0) |
| F10 | 6.0 | 20 mM Histidine- | 100 mM ArgGlu + 70 mM |
| | | Histidine HCl | trehalose |
| F11 | 6.0 | 20 mM Histidine- | 100 mM ArgHCl + 70 mM |
| | | Histidine HCl | trehalose |
| F12 | 6.0 | 20 mM Histidine- | 100 mM ArgHCl + 100 mM |
| | | Histidine HCl | Proline |

### Example 2: Viscosity Reducing Effect of Arginine Salts in High Concentration PD-1 Antibody Formulations

This set of experiments determined the viscosity results of formulations 1-12 with the varying protein concentrations described in Example 1. In the present example, we investigated the viscosity reducing effect of arginine salts, proline, combinations of arginine salts and polyols, combinations of arginine and proline and other organic electrolytes on the variables of viscosity and antibody stability.

Viscosity analysis was performed at a flow rate of 500 S⁻¹ and at the temperature of about 25° C. Results are shown in Table 3. This data demonstrated that the viscosity of Tislelizumab formulations was highly dependent on protein concentration. Viscosity increased exponentially when the protein concentration was increased from 160.42 mg/mL to 199.29 mg/mL in absence of viscosity reducer (F1). In absence of viscosity reducer, the viscosity values were 29.69 cP at a Tislelizumab protein concentration of 173.08 mg/mL and 53.29 cP at an antibody concentration of 199.29 mg/mL. In an unexpected result, addition of arginine salts such as ArgGlu, ArgHCl and ArgAsp significantly decreased the viscosity of high concentration Tislelizumab antibody formulations. When an arginine salt was present, viscosity values were 22.88 cP (189.47 mg/mL) and 30.62 cP (197.92 mg/mL) in F2 (ArgGlu), 21.24 cP (185.60 mg/mL) and 27.95 cP (191.70 mg/mL) in F3 (ArgHCl) and 20.70 cP (186.87 mg/mL) and 40.99 cP (211.33 mg/mL) in F4 (ArgAsp). At a Tislelizumab concentration of approximately 200 mg/ml, the viscosity was reduced by about 42.5% by ArgGlu. The results for these formulations are summarized in Table 3 below.

We also evaluated the impact of proline (F5) and other organic electrolytes such as NaGlu (F7) and LysHCI (F6) in the various Tislelizumab formulations. Results demonstrated that proline (F5) and NaGlu (F7) did not significantly reduce the viscosity, while a reduction in viscosity was observed for LysHCI (F6). However, the viscosity reducing effect of LysHCI was less significant than ArgGlu, ArgHCl and ArgAsp as found in F2-F4. The results for these formulations are summarized in Table 3 below.

Formulations 8 and 9 evaluated the viscosity reduction effects of arginine in the presence of an acetate counterion (e.g., arginine acetate (F8)) or a citrate counterion (e.g., arginine citrate (F9)). Arginine acetate and arginine citrate both produced viscosity reduction in Tislelizumab formulations, with arginine acetate producing a larger viscosity reduction than arginine citrate. However, the viscosity reduction produced by the presence of arginine acetate or arginine citrate was not as prominent as ArgGlu, ArgHCl and ArgAsp as found in F2-F4. The results for these formulations are summarized in Table 3 below.

Formulations 10, 11 and 12 investigated the impact of arginine salts and neutral organics combinations such as trehalose and proline. Results indicated that significant viscosity reductions were observed with 100 mM ArgHCl and 70 mM Trehalose combination (F11), 100 mM ArgGlu and 70 mM Trehalose combination (F10) and 100 mM ArgHCl and 100 mM Proline combination (F12). In summarizing these results, the viscosity decrease caused by 100 mM arginine salts and neutral organics combinations found in formulations F10-F12 was slightly less than 140 mM arginine salts alone.

In conclusion, the excipients that had the greatest impact on the viscosity of high concentration Tislelizumab formulations include the ArgGlu, ArgHCl and ArgAsp based formulations found in F2-F4.

TABLE 3
Viscosity of high concentration PD-1 antibody
formulations containing different excipients

| | Tislelizumab | Viscosity |
|---|---|---|
| Formulation | Concentration (mg/mL) | at 25° C. (cP) |
| F1 | 160.42 | 16.53 |
| | 173.08 | 29.69 |
| | 199.29 | 53.29 |
| F2 | 166.45 | 13.45 |
| | 189.47 | 22.88 |
| | 197.92 | 30.62 |
| F3 | 168.18 | 11.78 |
| | 185.60 | 21.24 |
| | 191.70 | 27.95 |
| F4 | 155.49 | 10.65 |
| | 186.87 | 20.70 |
| | 211.33 | 40.99 |
| F5 | 160.98 | 22.24 |
| | 183.35 | 31.34 |
| | 199.36 | 55.81 |
| F6 | 159.99 | 17.08 |
| | 181.38 | 24.30 |
| | 200.56 | 37.96 |
| F7 | 165.35 | 23.86 |
| | 183.42 | 37.37 |
| F8 | 169.31 | 18.22 |
| | 180.16 | 23.24 |
| | 200.46 | 41.53 |
| F9 | 166.66 | 21.37 |
| | 174.28 | 24.55 |
| | 190.63 | 32.52 |
| F10 | 158.36 | 13.03 |
| | 174.28 | 18.28 |
| | 197.03 | 32.30 |
| F11 | 169.06 | 16.20 |
| | 185.90 | 26.41 |
| | 199.07 | 39.43 |
| F12 | 168.83 | 16.04 |
| | 179.47 | 20.12 |
| | 191.27 | 32.72 |

### Example 3: Impact of pH and Buffer on the Viscosity of High Concentration PD-1 Antibody Formulations

This example evaluated the impact of pH and different buffers on viscosity in high concentration of Tislelizumab formulations containing arginine salts. The formulations are summarized in Table 4. The preparation of high concentration of Tislelizumab antibody was described above in Example 1. Briefly, Tislelizumab antibody stock solutions in 20 mM Histidine-Histidine HCl at pH6.5, pH6.0 and pH5.5 and in 20 mM Acetate at pH5.5 and pH5.0 were prepared by dialysis with 10 kDa MWCO dialysis cassettes. Stock solutions of viscosity lowering agent (without polysorbate 80 (PS80)) were spiked into Tislelizumab stock solutions, and then the samples were concentrated to about 200 mg/mL with 30 kDa Amicon Ultra™ centrifugal filters. Subsequently, high concentration PS80 solutions were added to achieve the target concentrations of 0.02%. Finally, formulations with varying protein concentrations were made by dilution with 1× stock solutions of viscosity lowering agent containing 0.02% PS80.

The viscosity of formulations F13-F18 containing ArgGlu and ArgHCl at a range of Tislelizumab concentrations is summarized in Table 5. The results indicated that in formulations containing arginine salts, lowering the pH from 6.5 to 5.0 significantly lowered the viscosity. At pH 5.5, the viscosity values of formulation F17 was 15.61 cP at an antibody concentration of 183.05 mg/mL, F18 was 16.32 cP at an antibody concentration of 183.17 mg/mL and F19 was 16.68 cP at an antibody concentration of 178.60 mg/ml. At pH 5.0, the viscosity values of F20 were 18.66 cP at an antibody concentration of 185.68 mg/mL and 24.74 cP at 199.95 mg/mL. Notably, no significant changes were observed in the viscosity of formulations containing acetate buffer compared with histidine buffer. In other words, changing buffer might not affect the viscosity of Tislelizumab antibody formulations containing arginine salts.

Subcutaneous formulations have an element of “syringe-ability” which is the ability of the subcutaneous formulation to be administered through a syringe (e.g., 20-25 gauge) needle. Specifically, the viscosity generated by high concentration Tislelizumab formulations showed good compatibility with a syringe containing a 23 or 25-gauge needle normally used for subcutaneous administration. Thus, the protein concentration of high Tislelizumab antibody formulations of F17, F18, F19 and F20 can be as high as 180-200 mg/mL with acceptable viscosity for subcutaneous delivery by syringe.

TABLE 4
Formulation Compositions

| Formu- | | | | |
|---|---|---|---|---|
| lation | | | | |
| Number | pH | Buffer | Viscosity Reducer | Surfactant |
| F13 | 6.5 | 20 mM Histidine- | 140 mM Arginine + | 0.02% |
| | | Histidine HCl | 140 mM Glutamic acid | polysorbate 80 |
| F14 | 6.5 | 20 mM Histidine- | 140 mM ArgHCl | 0.02% |
| | | Histidine HCl | | polysorbate 80 |
| F15 | 6.0 | 20 mM Histidine- | 140 mM Arginine + | 0.02% |
| | | Histidine HCl | 140 mM Glutamic acid | polysorbate 80 |
| F16 | 6.0 | 20 mM Histidine- | 140 mM ArgHCl | 0.02% |
| | | Histidine HCl | | polysorbate 80 |
| F17 | 5.5 | 20 mM Histidine- | 140 mM Arginine + | 0.02% |
| | | Histidine HCl | 140 mM Glutamic acid | polysorbate 80 |
| F18 | 5.5 | 20 mM Histidine- | 140 mM ArgHCl | 0.02% |
| | | Histidine HCl | | polysorbate 80 |
| F19 | 5.5 | 20 mM Acetate | 140 mM Arginine + | 0.02% |
| | | | 140 mM Glutamic acid | polysorbate 80 |
| F20 | 5.0 | 20 mM Acetate | 140 mM Arginine + | 0.02% |
| | | | 140 mM Glutamic acid | polysorbate 80 |

TABLE 5
Solution viscosity of PD-1 antibody formulations
with different pH and buffers

| | Tislelizumab | |
|---|---|---|
| | Concentration | Viscosity at |
| Formulation | (mg/mL) | 25° C. (cP) |
| F13 | 179.32 | 25.23 |
| | 202.48 | 46.26 |
| F14 | 179.44 | 28.21 |
| | 194.00 | 40.98 |
| F15 | 197.42 | 31.34 |
| F16 | 185.00 | 20.85 |
| F17 | 183.05 | 15.61 |
| | 194.52 | 27.71 |
| | 229.14 | 63.88 |
| F18 | 183.17 | 16.32 |
| | 196.04 | 31.77 |
| | 216.19 | 40.43 |
| F19 | 178.60 | 16.68 |
| | 194.48 | 41.73 |
| F20 | 185.68 | 18.66 |
| | 199.95 | 24.74 |

### Example 4: Impact of Varying Concentrations of Arginine Salts on the Viscosity of High Concentration PD-1 Antibody Formulations

These experiments tested varying concentrations of arginine salts on the viscosity of high concentration anti-PD-1 antibody formulations. To evaluate the concentration of arginine salts on viscosity, Tislelizumab antibody was formulated into 20 mM Histidine pH5.5, 0/50/100/280 mM arginine salts and 0.2 mg/ml polysorbate 80 (PS80). The preparation method was used as described in Examples 1 and 3.

Viscosity and osmolality were measured. Results are summarized in Table 6. Viscosity as well as osmolality of Tislelizumab antibody formulations with 0, 50, 100, 140 and 280 mM ArgHCl or ArgGlu at a range of antibody concentrations was tested. The results demonstrated that the addition of 50 mM ArgHCl or ArgGlu significantly decreased the viscosity of Tislelizumab antibody formulations. Additionally, incrementally increasing the concentration of ArgHCl or ArgGlu up to 280 mM further lowered the viscosity.

The experimentally determined osmolality values for each of these formulations are also listed in Table 6. The osmolality values of 140 mM ArgGlu and 140 mM ArgHCl formulations were between 360 and 390 mOsmol/kg, which was slightly hypertonic. Ideally, injectable products should be formulated as isotonic solutions (osmolality of about 300 mOsm/kg). However, strict isotonicity was not absolutely necessary for subcutaneous (SC) injection. It is recommended that the upper osmolarity limit should be controlled under 600 mOsmol/kg for drug product intended for subcutaneous injection to minimize hypertonicity-induced pain. Thus, the administration of a formulation containing higher concentration arginine salts (140 mM) demonstrated herein, for reducing the viscosity of the formulation, does not appear to present a risk of tissue damage at the injection site.

TABLE 6
Viscosity of high concentration PD-1 antibody formulations
containing varying concentrations of arginine salts

| | Antibody | | |
|---|---|---|---|
| | Concentration | Viscosity at | Osmolality |
| Arginine Salts | (mg/mL) | 25° C. (cP) | (mOsmol/kg) |
| none | 174.46 | 24.26 | NA |
| | 192.63 | 35.26 | |
| | 214.53 | 57.50 | |
| 50 mM ArgGlu | 176.63 | 18.57 | 195 |
| | 194.25 | 27.43 | 194 |
| 50 mM ArgHCl | 174.63 | 18.45 | 185 |
| | 192.76 | 28.63 | 184 |
| 100 mM ArgHCl | 179.90 | 16.86 | 257 |
| | 212.02 | 38.41 | NA |
| 140 mM ArgGlu | 183.05 | 15.61 | 366 |
| | 194.52 | 27.71 | 383 |
| | 229.14 | 63.88 | NA |
| 140 mM ArgHCl | 183.17 | 16.32 | 363 |
| | 196.04 | 31.77 | NA |
| | 216.19 | 40.43 | |
| 280 mM ArgGlu | 180.47 | 17.29 | 667 |
| | 193.53 | 25.64 | 693 |
| | 224.01 | 45.86 | NA |
| 280 mM ArgHCl | 183.25 | 17.83 | 580 |
| | 195.64 | 21.66 | 609 |
| | 221.84 | 38.77 | NA |

### Example 5: Evaluation of PD-1 Antibody Stability in Low-Viscosity and High Concentration Formulations

A further study was performed to evaluate the stability of formulations comprising about 180-200 mg/mL Tislelizumab antibody. In this study, arginine hydrochloride (ArgHCl), arginine glutamate (ArgGlu) and arginine aspartate (ArgAsp) were used as viscosity reducer, and the impact on stress and storage stability was evaluated.

For this study, Tislelizumab antibody was formulated into 20 mM Histidine/Acetate pH5.5, 140 mM arginine salts as shown in Example 1 in the preparation of formulations F2-F7. Subsequently, high concentration PS80 solutions were added to achieve the target concentrations of 0.05%. Each of the formulated solutions was filtered using Millex™ GP 0.22 μm PES 33 mm filter and filled into 2 mL ready-to-use glass vials (Schott), with a 0.5 mL drug product fill volume. Samples were staged, protected from light, and placed in a 2-8° C., 25° C. and 40° C. environmental stability chambers. The freeze-thaw stability was determined by stressing the formulations to three freeze/thaw cycles.

Turbidity of the drug product was determined on stability by measuring optical density at 350 nm. There were no significant changes in turbidity at 5° C. and 40° C. as well as under freeze-thaw condition for any of the formulations.

Purity of the formulations was determined by SEC-HPLC. At 40° C., there was an increase in aggregates, and a corresponding decrease in monomer was observed. Formulation F23 showed the most changes as compared to the other formulations. However, these reductions were considered acceptable for liquid formulations with intended storage conditions of 5° C. There were no changes in SEC purity at 5° C. and after freeze/thaw stress. At 25° C., about 0.4%-0.6% increase in aggregates was detected among formulations F21-F23 over 6 months.

Charge heterogeneity was detected by CZE and evaluated by monitoring the main peak along with acidic and basic species. At 5° C. up to 6 months, no measurable changes were found in any of the individual peaks including the main peak for any of the formulations. At 25° C. for 6 months, only a slight decrease was observed in main peak. At 40° C. for 4 weeks, the main peak showed a marked decline, indicating less antibody stability. Similar to the results observed at 25° C., increase was observed for acidic variants and decrease was observed in basic peaks (data not shown).

Purity by CE-SDS was measured under non-reducing conditions. There was no measurable change in purity as a functional of time at 5° C. up to 6 months for any of the formulations. The purity % at 5° C., 25° C. and 40° C. were all >96% and within the clinical acceptance criteria of >90.0% for non-reducing CE-SDS.

These results demonstrated that these formulations comprising about 180-200 mg/mL Tislelizumab antibody were stable after 3 freeze/thaw cycles and after 6 months storage at 5° C. and 25° C. and are summarized in Tables 7-9.

TABLE 7
Formulation Compositions Used in Stability Studies

| | Antibody | |
|---|---|---|
| Formulation | Concentration | |
| Number | (mg/mL) | Formulation Composition |
| F21 | 179.33 | 20 mM Histidine-Histidine HCl, 140 mM ArgGlu, 0.05% |
| | | polysorbate 80, pH 5.5 |
| F22 | 182.12 | 20 mM Acetate, 140 mM ArgAsp, 0.05% polysorbate 80, pH 5.5 |
| F23 | 201.73 | 20 mM Histidine-Histidine HCl, 140 mM ArgHCl, 0.05% |
| | | polysorbate 80, pH 5.5 |

TABLE 8
Stability Studies Scheme

| | Study Conditions | Test Items |
|---|---|---|
| High temperature | Samples were placed in a 40° C. | Turbidity (OD_{350 nm}) |
| (40° C.) study | stability chamber for 4 weeks | Purity (SEC and CE-SDS(NR) |
| Accelerated stability | Samples were placed in a 25° C. | Charge variants (CZE) |
| (25° C.) study | stability chamber for 6 months | |
| Long-term stability | Samples were placed in a 2-8° C. | |
| (2-8° C.) study | stability chamber for 6 months | |
| Freeze-thaw study | Three cycles of freezing at −40° C. and | |
| | thawing at ambient temperature | |

TABLE 9
Turbidity Data for Formulations F21-F23

| | Formulation | Initial | 40C4W | 3FT | 25C3M | 5C3M | 25C6M | 5C6M |
|---|---|---|---|---|---|---|---|---|
| Turbidity | F21 | 0.086 | 0.115 | 0.095 | 0.135 | 0.117 | 0.114 | 0.114 |
| (OD_{350 nm}) | F22 | 0.091 | 0.118 | 0.095 | 0.133 | 0.113 | 0.115 | 0.093 |
| | F23 | 0.093 | 0.118 | 0.104 | 0.150 | 0.122 | 0.142 | 0.125 |

Note:
40C4W refers to the samples stored at 40° C. for 4 weeks. 3FT refers to the samples subjected to 3 freeze/thaw cycles. 25C3M and 25C6M refers to the samples stored at 25° C. for 3 months and 6 months respectively. 5C3M and 5C6M refers to the samples stored at 5° C. for 3 months and 6 months respectively.

1. A low viscosity pharmaceutical formulation comprising:
about 10 mg/mL to about 200 mg/mL of an anti programmed death receptor 1 PD-1 antibody, or antigen binding fragment thereof;
a formulation buffer providing a pH of about 5.0 to about 7.0;
a viscosity reducer; and
a non-ionic surfactant,
wherein mentioned formulation has a viscosity of no more than 35 cP, and an osmolarity from about 200 mOsmol/kg to about 400 mOsmol/kg.

2. The formulation of claim 1, wherein the PD-1 antibody or antigen binding fragment thereof, comprises (a) a HCDR1 (Heavy Chain Complementarity Determining Region 1) of SEQ ID NO: 1, (b) a HCDR2 of SEQ ID NO:2, (c) a HCDR3 of SEQ ID NO:3 and a light chain variable region that comprises: (d) a LCDR1 (Light Chain Complementarity Determining Region 1) of SEQ ID NO:4, (e) a LCDR2 of SEQ ID NO:5, and (f) a LCDR3 of SEQ ID NO:6.

3. The formulation of claim 2, wherein the formulation buffer is selected from the group consisting of histidine, acetate, citrate, succinate, phosphate, mixture of histidine and acetic acid, or mixture of histidine and citric acid.

4. The formulation of claim 3 wherein the formulation buffer is histidine.

5. The formulation of claim 3 wherein the formulation buffer is acetate.

6. The formulation of claim 4, wherein:
(a) the concentration of buffer is 15 mM to 25 mM; or
(b) the formulation comprises 20 mM histidine buffer or 20 mM acetate buffer.

7. (canceled)

8. The formulation of claim 5, wherein the pH is 5.0-6.0.

9. The anti-human PD-1 antibody formulation of claim 1, wherein the viscosity reducer is an arginine salt.

10. The formulation of claim 9, wherein the arginine salt:
(a) is an equal mixture of L-arginine and L-glutamic acid (ArgGlu) from 50 mM to 280 mM;
(b) is an L-arginine and L-glutamic acid complexed salt (ArgGlu) from 50 mM to 280 mM;
(c) is an equal mixture of L-arginine and L-aspartic acid (ArgAsp) from 50 mM to 280 mM;
(d) is an L-arginine and L-aspartic acid complexed salt (ArgAsp) from 50 mM to 280 mM; or
(e) is L-arginine hydrochloride (ArgHCl) from 50 mM to 280 mM.

11. (canceled)

12. (canceled)

13. (canceled)

14. (canceled)

15. The formulation of claim 1, wherein the non-ionic surfactant is selected from the group consisting of polysorbate 20, polysorbate 80 or poloxamer188.

16. The formulation of claim 15, wherein the concentration of polysorbate 80 is from 0.02% to 0.08%.

17. The formulation of claim 16, wherein polysorbate 80 concentration is 0.05%.

18. The formulation of claim 1, wherein the formulation comprises:
(a) 20 mM Histidine-Histidine HCl, 140 mM ArgGlu, 0.05% polysorbate 80 with a pH of pH5.5;
(b) 20 mM Acetate, 140 mM ArgAsp, 0.05% polysorbate 80 with a pH of pH5.5; or
(c) 20 mM Histidine-Histidine HCl, 140 mM ArgHCl, 0.05% polysorbate 80 with a pH of pH5.5.

19. (canceled)

20. (canceled)

21. The formulation of claim 1, wherein the concentration of the anti-human PD-1 antibody, or antigen binding fragment thereof is from about 100 mg/mL to 200 mg/mL.

22. A method for treating cancer in a human patient in need thereof comprising subcutaneous administration of an effective amount of an anti-human PD-1 antibody formulation of claim 1.

23. The method of claim 22, wherein the anti-human PD-1 antibody formulation is administered:
(a) at a dose of about 100 mg to about 1000 mg;
(b) at a dose of 200 mg;
(c) at a dose of 300 mg;
(d) at a dose of 400 mg; or
(e) at a dose of 500 mg.

24. (canceled)

25. (canceled)

26. (canceled)

27. (canceled)

28. The method of claim 23, wherein the anti-human PD-1 antibody formulation is subcutaneously administered:
(a) once every three weeks;
(b) once every week;
(c) once every two weeks;
(d) once every three weeks.

29. (canceled)

30. (canceled)

31. (canceled)

32. The method of claim 23, wherein the cancer is lung cancer (including small-cell lung cancer, or non-small cell lung cancer), adrenal cancer, liver cancer, stomach cancer, cervical cancer, melanoma, renal cancer, breast cancer, colorectal cancer, leukemia, bladder cancer, bone cancer, brain cancer, an endometrial cancer, head and neck cancer, lymphoma, ovarian cancer, skin cancer, thyroid tumor, or esophageal cancer.

33. The method of claim 23, wherein the human patient is administered at least one other therapeutic agent.

34. The method of claim 33, wherein the at least one other therapeutic agent is zanubrutinib, pamiparib, an anti-CTLA4 antibody, an anti-4-1BB antibody, an anti-OX40 antibody, an anti-TIGIT antibody, an anti-TIM-3 antibody, a second PD-1 antibody, a CD40 agonist, a TLR agonist, a CAR-T cell, or a chemotherapeutic agent.


SEQUENCE LISTING (11 sequences)

SEQ ID NO: 1 - protein, 10 aa, synthetic construct
GFSLTSYGVH

SEQ ID NO: 2 - protein, 16 aa, synthetic construct
VIYADGSTNYNPSLKS

SEQ ID NO: 3 - protein, 12 aa, synthetic construct
ARAYGNYWYIDV

SEQ ID NO: 4 - protein, 11 aa, synthetic construct
KSSESVSNDVA

SEQ ID NO: 5 - protein, 7 aa, synthetic construct
YAFHRFT

SEQ ID NO: 6 - protein, 9 aa, synthetic construct
HQAYSSPYT

SEQ ID NO: 7 - protein, 118 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 8 - protein, 107 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 9 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 10 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 11 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC